(19) United States
(12) Patent Application Publication
(10) Pub. No.: US 2019/0262488 A1
(43) Pub. Date: Aug. 29, 2019

(54) SYSTEM AND METHOD FOR IN-PLACE MEAT GRINDER SANITIZATION

(71) Applicant: THE BOARD OF REGENTS FOR OKLAHOMA STATE UNIVERSITY, STILLWATER, OK (US)

(72) Inventors: RAVIRAJSINH JADEJA, Stillwater, OK (US); YEN-CON HUNG, PEACHTREE CITY, GA (US)

(73) Assignee: THE BOARD OF REGENTS FOR OKLAHOMA STATE UNIVERSITY, STILLWATER, OK (US)

(21) Appl. No.: 16/409,069

(22) Filed: May 10, 2019

Related U.S. Application Data
(63) Continuation of application No. 15/060,163, filed on Mar. 3, 2016, now abandoned.
(60) Provisional application No. 62/143,430, filed on Apr. 6, 2015.

(51) Int. Cl. A61L 2/23; A01N 59/00; A61L 2/16

(52) U.S. Cl. CPC A61L 2/23; A61L 2/16; A01N 59/00

(57) ABSTRACT

According to an embodiment, there is provided herein a method of using electrolyzed oxidizing water ice (EO ice) as a Cleaning-In-Place (CIP) method that reduces antimicrobial contamination. One embodiment is utilized in connection with beef grinders. In one embodiment, EO ice was prepared by freezing 200 mg/L free chlorine-containing EO water at 20° C. overnight. The EO ice and, optionally, EO ice/water mixture was then ground within an operating meat grinder. The EO ice treatment in combination with EO water has the potential to reduce cross-contamination and could serve as an easy to apply antimicrobial intervention to improve overall safety of ground beef.

### CROSS REFERENCE TO RELATED APPLICATIONS

This application is a continuation of co-pending U.S. application Ser. No. 15/060,163, filed Mar. 3, 2016, which application claims the benefit of U.S. Provisional Patent Application Ser. No. 62/143,430 filed on Apr. 6, 2015, and incorporates said applications by reference into this document as if fully set out at this point.

### TECHNICAL FIELD

This disclosure relates generally to methods of sanitization and, more particularly, to systems and methods of sanitizing food preparation equipment such as meat grinders.

### BACKGROUND

Shiga toxin-producing E. coli (STEC), and Salmonella enterica are two major groups of foodborne pathogens in the United States. STEC infections are responsible for approximately 2409 hospitalizations yearly and among these cases, over 2100 cases are caused by E. coli O157: H7. Various Salmonella spp. are responsible for 1,027,561 illnesses and 19,336 hospitalizations each year in the United States.

Cattle are a well-known source of E. coli O157: H7 and Salmonella enterica and because of that, beef products carry a significant risk of contamination with these foodborne pathogens. The muscles of a healthy animal are free of pathogens, but during slaughtering and especially during hide removal process, pathogens may get transferred to the surface of the carcass and may cause consumer illness if not handled properly. Beef products, especially ground beef, are notoriously associated with foodborne outbreaks. Ground beef is usually prepared from beef trimmings that are generally obtained from the surfaces of whole muscle cuts during fabrication and hence, has higher possibilities to contain pathogens.

Some of the contamination events occurring during beef processing may be the result of cross-contamination by knives and other food contact surfaces. There are existing protocols and control points in the meat processing or retail operations, which specify the frequency and proper procedures of grinder sanitization. However, if contamination occurs between two cleaning operations, grinder will potentially cross-contaminate large amount of products. An increase in the frequency of dissembling the grinder for cleaning will lead to increase in operation cost and reduced productivity. Hence, there is a need to develop an intervention step to controlling cross-contamination while grinding.

Electrolyzed oxidizing water is (EO water) known for its antimicrobial efficacy. EO water has been proven effective in reducing variety of foodborne pathogens from various food matrices and food contact surfaces. Several studies have also reported that the use of EO water ice (EO ice) can significantly improve overall microbiological quality of seafood. It has been reported that when Pacific saury IS stored in BO ice containing 47 mg/L available chlorine, its shelf life is extended up to 5 days and observed significantly reduce the growth of aerobic and psychrotrophic bacteria on the fish. It has also been reported that an EO ice (100 mg/L available chlorine) treatment for 24 h was able to reduce E. aerogenes and M. morganii on tuna skin by 2.4 and 3.5 log CFU/cm², respectively.

Heretofore, as is well known in the food preparation arts there has been a need for an invention to that is designed to overcome the disadvantages of prior art approaches. Accordingly it should now be recognized, as was recognized by the present inventors, that there exists, and has existed for some time, a very real need for a system that would address and solve the above-described and other problems.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

There is provided herein a single step antimicrobial ice-based meat grinder sanitation process. This method could utilize ice prepared from any sanitizer approved for food contact surface use and provides a significant reduction in foodborne pathogens that can be found on a meat grinder of the sort used to process beef, chicken, pork, etc.

According to one embodiment, there is taught herein an antimicrobial electrolyzed oxidizing water ice based (EO ice) Cleaning-In-Place (CIP) method that has been developed and optimized for beef grinders. EO ice was prepared by freezing free chlorine-containing EO water at −20° C. overnight. Although other temperatures might be used to prepare the EO ice, EO ice at this temperature produced an acceptable amount of physical abrasion which is useful in removing meat particles from the body of the grinder. Generally speaking, lower temperatures would produce harder ice that, in turn, would produce more abrasion when it is processed within the meat grinder.

The EO ice is then placed into a contaminated grinder and the grinder activated. The abrasive and disinfective action of the EO ice significantly reduced the number of pathogens present in the grinder.

Some of the aspects of one embodiment taught herein are:
(1) An amount of EO ice (e.g., 250, 500 and 1000 g) sufficient to reduce Escherichia coli O157:H7 from inoculated meat grinders was prepared;
(2) Antimicrobial potential of various combinations of EO ice and EO water −150 mg/L free chlorine (1000 g EO ice+200/400 or 600 ml EO water) to reduce pathogens from inoculated meat grinder were prepared.
(3) The EO ice was passed through the grinder by operating it as usual.
(4) The efficacy of an EO ice/EO water treatment to reduce E. coli O157:H7 or Salmonella typhimurium DT 104 from the meat grinders inoculated by processing beef was determined to be approximately 6 or 3 log CFU/g pathogen reduction.

Continuing with the previous example, in the embodiments set out above five 200 g uninoculated beef pieces were ground and collected after each treatment. Efficacies of EO ice based treatments were compared with Deionized water ice (DI ice) and no treatment controls. EO ice, DI ice and no treatment control treatments yielded E. coli O157:H7 recoveries ranging from 4.05 to 1.92, 4.32 to 2.76 and 5.40 to 3.12 log CFU/g from ground beef samples 1 to 5, respectively. EO ice and EO water combination treatments further decreased E. coli O157: H7 and after 1000 g EO ice with 600 ml EO water reduced the cross-contamination in all ground beef samples with the E. coli O157:H7 and recoveries ranging from 2.43 to <1 log CFU/g were obtained. In the last part, when grinders were inoculated with low levels of pathogens, 1000 g EO ice+600 ml EO water treatment eliminated E. coli O157: H7 and S. typhimurium DT 104 cross-contamination. Recoveries from the grinders inoculated with higher level of pathogens were 3.52 to <1 and 3.06 to <1 log CFU/g in ground beef samples 1 to 5 for E. coli O157:H7 and S. typhimurium DT 104, respectively.

As such, the EO ice treatment, optionally in combination with some amount of EO water, has the potential to reduce cross-contamination and could serve as an easy to apply antimicrobial intervention to improve overall safety of ground beef which does not materially interrupt the meat production process.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1 contains a plot of efficacy of EO water ice to reduce low levels of E. coli O157: H7 from the meat grinder according to one embodiment. NT: no treatment, C: DI water ice 1000 g+600 ml DI water and I: EO water ice 1000 g+600 ml EO water. Measurements A-G with no common letter denote values that are significantly different (P≤0.05)

FIG. 2 contains a plot of efficacy of EO water ice to reduce low levels of S. typhimurium DT104 from the meat grinder according to an embodiment. NT: no treatment, C: DI water ice 1000 g+600 ml DI water and I: EO water ice 1000 g+600 ml EO water. Measurements A-F with no common letter denote values that are significantly different (P≤0.05).

FIG. 3 contains a plot of efficacy of EO water ice to reduce high levels of E. coli O157:1-17 from the meat grinder according to one embodiment. NT: no treatment, C: DI water ice 1000 g+600 ml DI water and I: EO water ice 1000 g+600 ml EO water. Measurements A-H with no common letter are significantly different (P≤0.05).

FIG. 4 contains a plot of efficacy of EO water ice to reduce high levels of S. typhimurium DT104 from the meat grinder based on one embodiment. NT: no treatment, C: DI water ice 1000 g+600 ml DI water and I: EO water ice 1000 g+600 ml EO water. Measurements A-H with no common letter denote values that are significantly different (P≤0.05).

FIG. 5 contains a table that illustrates the efficacy of EO water ice to reduce E. coli O157:H7 from directly inoculated beef grinder according to an embodiment. NT: no treatment, C1: Deionized water ice 250 g treatment, I1: EO water ice 250 g treatment, C2: Deionized water ice 500 g treatment, 12: EO water ice 500 g treatment, C4: Deionized ice 1000 g treatment, 14: EO water ice 1000 g treatment. The superscripts a-p with no common letter denote entries that are significantly different (P≤0.05) from each other.

FIG. 6 contains a table that illustrates the efficacy of EO water ice plus EO water to reduce E. coli O157:H7 from directly inoculated beef grinder. NT: No treatment, C4+2: Deionized water ice 1000 g+200 ml Deionized water treatment, I4+2: EO water ice 1000 g+200 ml EO water treatment, C4+4: Deionized water ice 1000 g+400 ml Deionized water treatment, I4+4: EO water ice 1000 g+400 ml EO water treatment, C4+6: Deionized water ice 1000 g+600 ml Deionized water treatment, I4+6: EO water ice 1000 g+400 ml BO water treatment. The superscripts a-r no common letter denote entries that are significantly different (P≤0.05) from each other.

FIG. 7 contains a plot of the efficacy of EO water ice in reducing high levels of E. coli O157: H7 from the meat grinder according to an embodiment. In this figure, NT stands for “no treatment”, I stands for DI water ice 1000 g+600 ml DI water, and PAA stands for peroxyacetic acid-based EO ice 1000 g+600 ml PAA.

FIG. 8 illustrates the efficacy of EO water ice to reduce low levels of E. coli O157: H7 from the meat grinder in connection with an embodiment. In this figure, NT stands for “no treatment”, I stands for DI water ice 1000 g+600 ml DI water, and PAA stands for peroxyacetic acid-based EO ice 1000 g+600 ml PAA

FIG. 9 contains an operating logic suitable for use with an embodiment.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

### Materials and Methods:

Inoculum Preparation:

According to this embodiment, a total of ten strains of E. coli O157: H7 and S. typhimurium DT 104 were used. The five strains of E. coli O157: H7 were 1 (Beef isolate), 5 (human isolate), 932 (human isolate), E009 (Beef isolate) and E0122 (cattle isolate); and five strains of Salmonella typhimurium DT104 were H2662 (cattle isolate), 11942A (cattle isolate), 13068A (cattle isolate), 152N17-1 (dairy isolate) and H3279 (human isolate). All E. coli O157: H7 strains were adapted to 50 mg/L nalidixic acid for ease of isolation. Before each experiment, E. coli O157:H7 strains were grown individually in tryptic soy broth (TSB; Difco, Becton Dickinson, Sparks, Md.) supplemented with 50 mg/L nalidixic acid and S. typhimurium DT 104 strains were grown in TSB supplemented with 32 mg/ml ampicillin, 16 mg/ml tetracycline and 64 mg/ml streptomycin. Each overnight grown strain was then washed by centrifugation (3,000×g for 15 min), and pellet was resuspended in phosphate buffered saline (PBS, pH-7). Two different five strain mixtures were prepared by mixing 2 ml of individual strains of respective pathogens. Appropriate dilutions were made using PBS to achieve final concentrations of approximately 9 log CFU/ml and (high inoculums) and 6 log CFU/ml (low inoculums).

Antimicrobial Ice Preparation:

EO water was produced by electrolyzing a dilute NaCl solution (0.03%) in EAU EO water generator (Model # P30HST44T, EAU, GA, USA). The pH and ORP of EO water were measured using an ACCUMET pH meter (AR50, Fisher Scientific, Pittsburgh, Pa.). The initial free chlorine concentration of samples was determined by a DPD-FEAS method (Hach Co., Loveland, Colo.). Appropriate dilutions were made to achieve final free chlorine concentration of approximately 200 mg/L in EO water. Then EO water ice was prepared by freezing EO water solutions in plastic ice trays at −20° C. overnight.

Inoculation Procedures:

Two different inoculation procedures were used in this study; 1) Direct inoculation of grinder and (direct) and 2) Inoculation through artificially contaminated meat (indirect). For the direct inoculation procedure, a bench top grinder (Model #781, LEM products, OK, USA) was used. Two approximately 200 g beef pieces (4″L×4″W, prepared from beef shoulder clods, temperature of beef—2.0±2 C.°) were ground to create food matrix inside grinder to simulate a regular grinding operation. At the end of grinding process, equipment was artificially contaminated by directly placing 200 μl bacterial cocktail on multiple places on the auger stud and grinder head in the form of ten 20 μl spots. Pathogens were allowed to attach to the surface for 30 min at room temperature. Indirect inoculation was carried out using following procedure. Two uninoculated 200 g beef pieces were ground to create food matrix inside the grinder as described earlier. The spiked beef pieces were prepared by inoculating 200 μl bacterial suspension on each beef piece. In order to contaminate grinder, three artificially contaminated beef pieces were processed through the grinder. Pathogens were allowed to attach for 30 min at room temperature.

Bacterial transfer and decontamination experiments: This study was divided into three phases. For the first two parts, optimum treatment conditions for reduction of E. coli O157: H7 from directly inoculated grinders were determined using a varying amount of EO ice (250, 500 and 1000 g) and EO ice with 150 mg/L free chlorine-containing EO water (1000 g ice+100, 200 or 400 ml EO water). In order to determine the antimicrobial efficacy of EO ice treatment in ‘real life’ scenario, in the third set of experiments, indirect inoculation protocol was employed to contaminate grinder artificially with E. coli O157:H7 and S. typhimurium DT 104 at high and low inoculation levels. The most effective treatment conditions identified from the first two experiments were used in the third set of experiments. After each treatment, five uninoculated beef pieces (approximately 200 g each) were processed through the grinder. Individual ground beef portions corresponding to beef pieces were collected in separate sterile stomacher bags (Seward, Worthing, UK). For all experiments, efficacies of EO ice based treatments were compared with Deionized water ice (DI ice) and no treatment controls.

Microbiological Analysis:

Ground beef corresponding to each piece grinded after treatment was mixed with neutralizing buffer (1:1 w/v) followed by mixing for 2 min using a stomacher. Further appropriate dilutions were made and 0.1 ml portions were plated on sorbitol MacConkey agar (SMA; Oxoid, Basingstoke, UK) supplemented with 50 mg/L nalidixic acid for E. coli O157:H7 or xylose lysine deoxycholate agar (XLD; Becton Dickinson, Sparks, Md.) supplemented with 32 mg/ml ampicillin, 16 mg/ml tetracycline, and 64 mg/ml streptomycin for S. typhimurium DT 104. Plates were stored at 37° C. for 24 h before counting. At the end of the incubation period, plates were observed for typical E. coli O157: H7 (colorless) and Salmonella (black) colonies. Selection and confirmation of E. coli O157: H7 and S. typhimurium DT 104 isolates were carried out using the procedure described in a previously published study. Briefly, typical colonies of E. coli O157: H7 and S. typhimurium DT 104 were streaked on SMA and XLD. E. coli O157: H7 colonies from SMA were tested for agglutination by E. coli O157 latex agglutination assay (Oxoid). Colonies that exhibited positive agglutination reaction were once again streaked on SMA and colonies were identified as E. coli using the API 20E test (bioMe'rieux, Hazelwood, Mo.). Further confirmative tests were carried out using Bacto E. coli O157 and H7 antisera. Similarly, Salmonella confirmation was carried out using Salmonella latex agglutination assay (Oxoid) and API 20E assay (bioMe'rieux). Presence of pathogens was also determined from ice samples after grinding and from ground beef sample that did not yield countable colonies through enrichment using the procedure described elsewhere.

Statistical Analysis:

All results presented are outcomes of at least three independent experimental replicates. Statistical analysis was performed using JMP PRO 11 (SAS Institute, Inc., Cary, N.C.). Tukey-Kramer test at the probability level of P≤0.05 was used for pairwise comparisons of means.

### Results and Discussion:

Reduction of E. coli O157: H7 from directly inoculated grinder after various antimicrobial ice treatments:

In the first set of experiments, varying amounts of EO ice was processed through directly contaminated grinder. Increasing the amount of ice (DI water ice or EO ice) resulted in a significant increase in pathogen removal from the inoculated grinders.

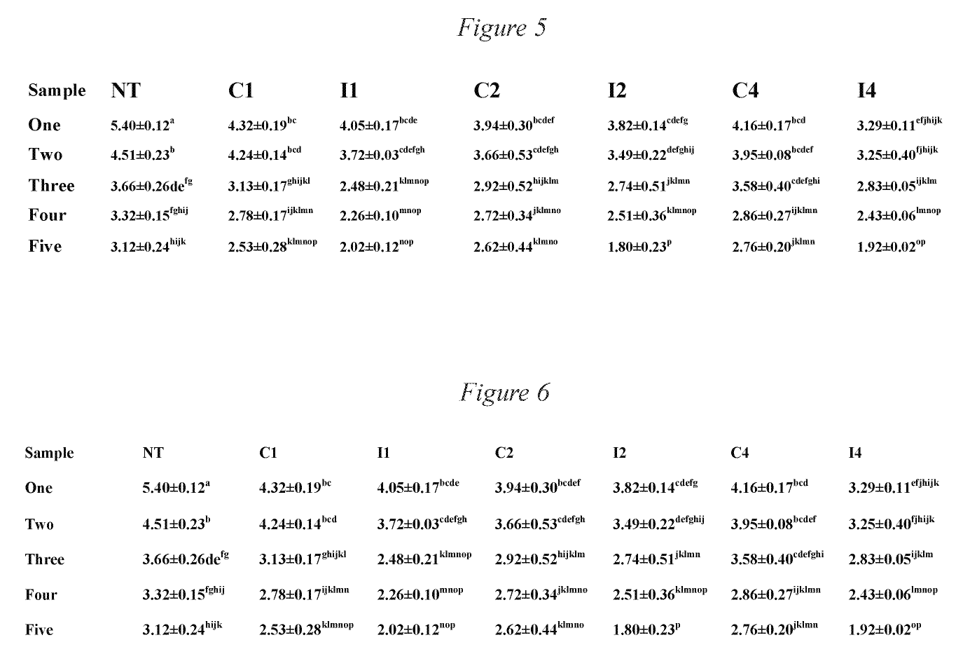

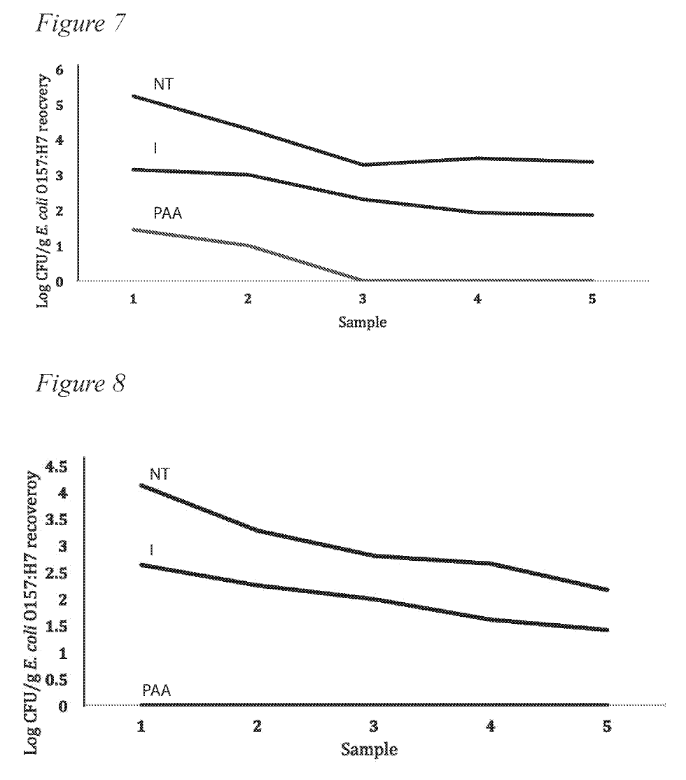

FIG. 5 shows the recovery of E. coli O157: H7 from directly inoculated grinder after various treatments. After processing five non-contaminated beef pieces through inoculated grinders without any treatment yielded recoveries of 5.40, 4.51, 3.66, 3.32 and 3.12 log CFU/g E. coli O157:H7 from ground beef samples one to five, respectively. When inoculated grinders were subjected to 1000 g EO ice treatment, pathogen recoveries reduced to 3.29, 3.25, 2.83, 2.43 and 1.92 log CFU/g for samples 1 to 5, respectively. DI ice treatments also, significantly reduce pathogens in comparison of no treatment control but, all DI water ice samples collected after processing were tested positive for E. coli O157: H7 while, all EO water ice samples tested negative for the target pathogen after enrichment (data not shown). More than 1000 g of ice led to freezing of grinder head and hence, based on pathogen removal capacities and visual observation of grinder cleanliness, it was decided to use 1000 g ice for rest of the study. In the second set of experiments, 1000 g DI or EO ice was mixed with 200, 400 or 600 ml of DI or EO water before processing. Similar pathogen reduction trend as of experiment one was observed in this experiment and the amount of EO water used, and pathogen recoveries were directly proportional to each other.

FIG. 6 exhibits the recoveries of E. coli O157: H7 after various treatments. Results indicate that first ground beef sample obtained without any treatment had 5.37 log CFU/g E. coli O157:H7 while, bacterial recovery reduced to 4.54 log CFU/g from the second ground beef sample and even at 5^{th} sample 3.28 log CFU/g E. coli O157: H7 were recovered. When 1000 g EO ice combined with 200 ml EO water, bacterial recoveries ranging from 3.52 to 2.31 log CFU/g were observed in ground beef samples 1 to 5. A declining trend of E. coli O157: H7 recoveries, 3.22 to 1.91 log CFU/g for samples one to five, was observed when EO water amount was increased to 400 ml in the treatment. Among all treatments, 1000 g EO ice with 600 ml EO water found most effective in removing targeted pathogens from the equipment and the transfer of E. coli O157:H7 from grinder to ground beef reduced to 2.43 log CFU/g (from the first sample) which was significantly less in comparison of 5.37 log CFU/g no treatment control. For the same treatment, no E. coli O157: H7 was recovered from the fifth sample through direct platting but, 24 h enrichment of the ground beef sample tested positive for the presence of the targeted pathogens. Because of the superior E. coli O157: H7 reduction capabilities of the combination treatment, EO ice+EO water (1000 g EO ice+600 ml EO water), it was decided to use this treatment for further studies.

In order to mimic the contamination conditions occur in nature, meat grinders were inoculated with target pathogens by processing artificially spiked meat pieces. Antimicrobial efficacy of the treatment was determined for grinders inoculated with high and low levels of pathogens.

For low levels of inoculation, 1000 g EO ice+600 ml EO water treatment eliminated the transfer of pathogens to ground beef (no pathogens were recovered from the first sample even after enrichment). While, bacterial recovery from the first piece processed for no treatment control and 1000 g DI ice+600 ml DI water were 2.76 and 1.64 log CFU/g in ground beef (FIG. 1), respectively. When grinders were treated with 1000 g DI ice+600 ml DI water it was not before 4^{th} ground beef sample that the transfer of E. coli O157:H7 to ground beef reduced to non-detectable levels by direct plating but, after enrichment ground beef samples four and five found positive for the targeted pathogen.

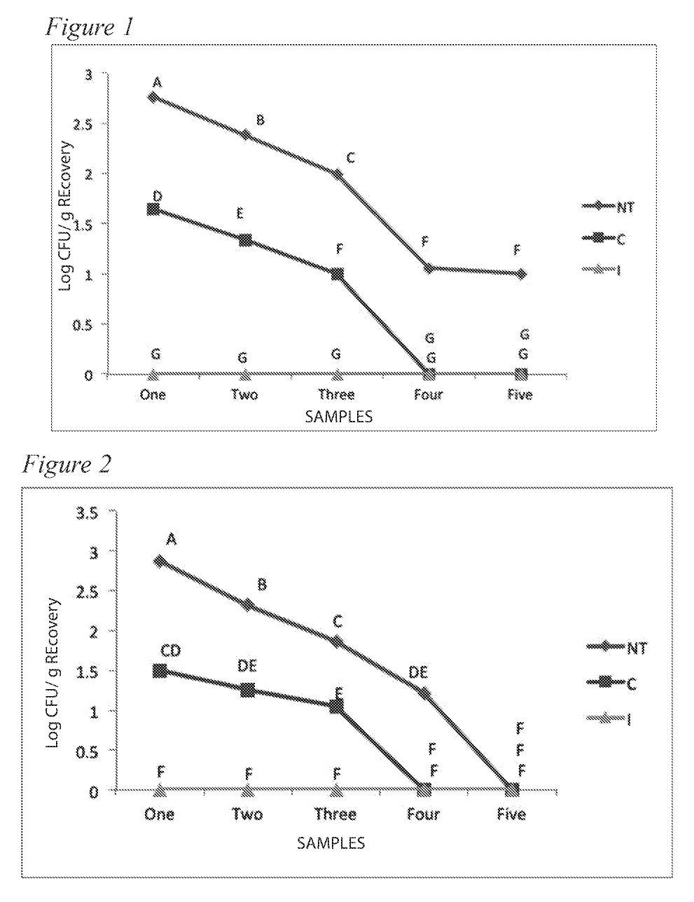

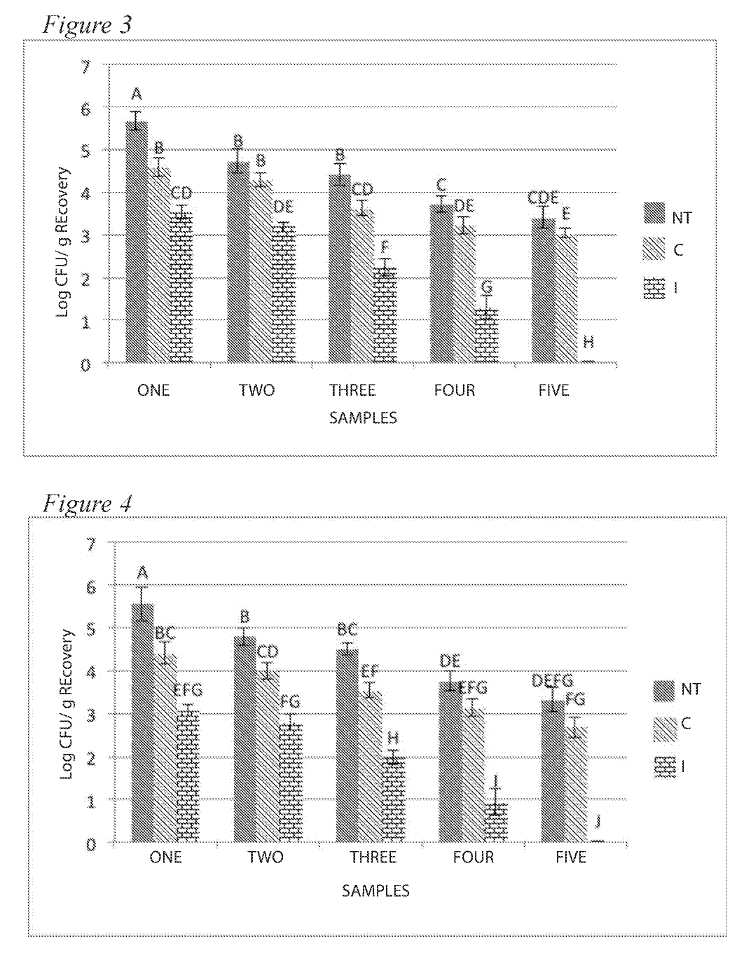

Similar trend of pathogen reduction from the grinders as of E. coli O157: H7 was observed when S. typhimurium DT 104 was inoculated to meat grinders through processing spiked beef pieces. After DI ice (1000 g)+DI water (600 ml) and no treatment control, 1.5 and 2.87 log CFU/g S. typhimurium DT 104 were recovered from first ground beef sample, respectively (FIG. 2). On the other hand, EO ice (1000 g)+EO water (600 ml) treatment eliminated cross-contamination of ground beef from the very first ground beef sample and all ground beef samples were free of targeted pathogens even after enrichment. The cross-contamination of S. typhimurium DT 104 for DI ice+DI water and no treatment control did not reduce to non-detectable levels by direct plating until ground beef samples four and five, respectively.

Effectiveness of EO water ICE and EO water treatments to reduce pathogens was also determined on meat grinders inoculated with high levels of E. coli O157: H7 and S. typhimurium DT 104. EO water ice with EO water treatment significantly reduced the cross-contamination of E. coli O157: H7 and S. typhimurium DT 104 in ground beef in comparison of no treatment control and DI ice+DI water treatments. For grinders inoculated with E. coli O157:H7, no treatment control, DI ice (1000 g)+DI water (600 ml) and EO ice (1000 g)+EO water (600 ml) yielded bacterial recoveries ranging from 5.66 to 3.39, 4.57 to 3.02 and 3.52 to <1 log CFU/g for samples one to five, respectively. Similarly, control, DI ice+water and EO ice+water treatments reduced S. typhimurium DT 104 levels to 3.30, 2.67 and <1 log CFU/g from the fifth ground beef sample from each treatment, respectively.

### Peroxyacetic Acid (PAA) Based Antimicrobials: Materials and Methods:

Inoculum Preparation:

In this example, a total of 5 strains of E. coli O157: H7 were used. All strains were adapted to 50 mg/L nalidixic acid for ease of isolation. Before each experiment, E. coli O157:H7 were grown individually in tryptic soy broth (TSB; Difco, Becton Dickinson, Sparks, Md.) supplemented with 50 mg/L nalidixic acid. Each overnight grown strain were then washed by centrifugation (3,000×g for 15 min), and pellet were resuspended in phosphate buffered saline (PBS, pH-7). A five strain mixtures was prepared by mixing 2 ml of individual strains of E. coli O157:H7. Appropriate dilutions were made using PBS to achieve final concentrations of approximately 9 log CFU/ml and (high inoculums) and 7 log CFU/ml (low inoculums).

Antimicrobial Ice Preparation:

PAA working solution was prepared as per manufacturers' instruction (230 mg/L PAA final concentration). PAA ice was prepared by freezing PAA solutions overnight in plastic trays.

Inoculation Procedures:

A bench top grinder (Model #781, LEM products, OK, USA) was used in this study. Two approximately 200 g beef pieces (4″L×4″W, from beef shoulder clods, temperature of beef—2.0±2 C.°) were grinded to create food matrix inside grinder to simulate a regular grinding operation. The spiked beef pieces were prepared by inoculating 200 μl bacterial suspension on each beef piece. In order to contaminate grinder, three artificially contaminated beef pieces were processed through the grinder. Pathogens were allowed to attach for 30 min at room temperature.

Bacterial Transfer and Decontamination Experiments:

The antimicrobial efficacy of PAA ice was determined by processing 1000 g PAA ice+600 ml PAA solution (230 mg/L) through contaminated meat grinder. At the end of PAA ice process, five uninoculated beef pieces (approximately 200 g each) were processed through the grinder. Individual ground beef portions corresponding to beef pieces were collected in separate sterile stomacher bags (Seward, Worthing, UK). Antimicrobial efficacies of PAA ice based treatments were compared with Deionized water ice (DI ice) and no treatment controls.

Microbiological Analysis:

Ground beef corresponding to each piece grinded after treatment were mixed with neutralizing buffer (1:1 w/v) followed by mixing for 2 min using a stomacher. Further appropriate dilutions were made and 0.1 ml portions was plated on sorbitol MacConkey agar (SMA; Oxoid, Basingstoke, UK) supplemented with 50 mg/L nalidixic acid. Plates were stored at 37° C. for 24 h before counting. At the end of the incubation period, plates were observed for typical E. coli O157: H7 (colorless)) colonies. Selection and confirmation of E. coli O157: H7 isolates were carried out using the procedure described in a previously published study. Briefly, typical colonies of E. coli O157: H7 were streaked on SMA. E. coli O157: H7 colonies from SMA were tested for agglutination by E. coli O157: H7 latex agglutination assay (Oxoid). Colonies that exhibited positive agglutination reaction will be once again streaked on SMA and colonies were identified as E. coli using the API 20E test (bioMe′rieux, Hazelwood, Mo.).

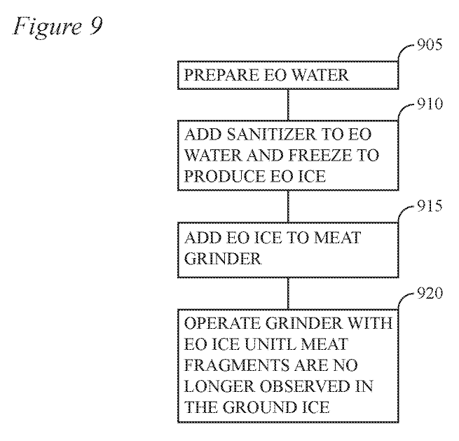

Turning next to FIG. 9, this figure contains an operating logic suitable for use with an embodiment. With respect to box 905, EO water will be prepared as described above or according to any other method known to those of ordinary skill in the art.

Box 910 indicates that the EO ice will be created by adding an amount of FDA approved sanitizer component to the EO water. Among the many sorts of sanitizers that would work in addition to free chlorine described herein are sanitizers such as hypochlorous acid (sodium salt), iodine, lactic acid, acetic acid, peroxyacetic acid, quaternary ammonium compounds, etc. Those of ordinary skill in the art will recognize that the foregoing are only examples of the sorts of sanitizers that might be used. Other examples can be found within the published FDA sanitizing guidelines found in 21 CFR 178.1010 and 40 CFR 180.940.

As to the amount of sanitizer that is to be added, in the case of free chlorine it might be in the range of about 50-200 mg/L, although other concentrations are certainly possible. Choice of the concentration might be based, among others, on the particular sanitizer used, etc.

As mentioned previously, it is preferred that the ice be of sufficient hardness to act as an abrasion when it is ground by the operating grinder. One example of a measurement of ice hardness is given by Energy Star® which is defined to be the latent heat capacity of harvested ice (in Btu/lb) divided by 144 Btu/lb and expressed as a percentage. Put simply, it is the percentage of harvested ice that is actually frozen ice. Ice flakes have hardness of approximately 70% (70% ice+30 chilled water trapped inside) while a transparent ice cube has a hardness of 100%. In some embodiments, an ice hardness suitable for use as disclosed herein might be between about 75% and 100%. In one example, a chilling temperature of −20° C. was adequate to reach this level of hardness, although clearly other temperatures (warmer or colder) might be used as well.

That being said, in order to get a preferred level of abrasion and quick sanitation when the EO ice is ground, use of 100% ice is recommended but even using, for example, 50% (or even less), 60%, 70%, 75%, 80%, or 90% ice it is possible to obtain significant pathogen reduction from meat grinders. Of course, lower levels of hardness will typically take longer to accomplish the same degree of cleaning and require a greater volume of ice to produce the same level of cleaning as that produced by the more abrasive/harder ice. Those of ordinary skill in the art will readily be able to determine (e.g., by trial and error) how long the grinder might need to be operated while giving it a supply of EO ice as a function of its hardness. In some embodiments, the EO ice prepared as described above will be continuously added to the grinder until such time as no meat fragments can be visually observed in the ground ice issuing from the meat grinder.

In that regard and according to box 915, the previously prepared BO ice will be added to a meat grinder and then it will be set into operation (box 920). The grinder might be operated until the ground EO ice material emerging from the grinder is visibly free of meat particles. In other cases, a specific time period might be specified (e.g., the grinding might proceed for 3 to 5 minutes). Of course, the length of time necessary to reduce the pathogens in a specific contaminated blender to an acceptable level might need to be determined empirically and/or by trial and error. Those of ordinary skill in the art will understand how this might be done.

Use of EO ice to improve microbiological quality of food products is well documented but this is the first known instance of developing a CIP process for meat grinders using EO ice. Current industry practices involve cleaning and sanitization of meat grinders at the end of the shift, and there is no intervention to control cross-contamination during processing. Further, such an approach involves five specified steps and might require 2 to 4 hours to complete.

One of the benefits of using EO ice is that it does not increase the temperature of the grinder unit. The temperature of the grinder is a very important parameter to control in order to maintain the ground product at a safe temperature. Besides temperature control, the cleaning/sanitizing effect of EO water ice could be functions of its antimicrobial capabilities and physical abrasion process.

Use of EO ice does not require disassembling of the grinder, therefore; this intervention could be easily applied during a shift to reduce microbial cross-contamination in ground beef products. The CIP process discussed herein could not only improve food safety by reducing the chances of cross-contamination but also dramatically reduce the size (quantity of product) of the recall.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A method of in-place sanitizing a meat grinder, comprising the steps of:
a. accessing a quantity of EO ice containing a sanitizing component therein;
b. loading at least a portion of said EO ice in said meat grinder; and
c. grinding said at least a portion of said quantity of EO ice within the meat grinder, thereby sanitizing the meat grinder in place.

2. The method according to claim 1, wherein said sanitizing component is free chlorine at a concentration of between 50 and 200 mg/L.

3. The method according to claim 1, wherein EO ice has a hardness of between 75% and 100%, where said hardness is measured as a latent heat capacity of said EO ice in Btu/lb divided by 144 Btu/lb and expressed as a percentage.

4. The method according to claim 1, wherein said sanitizing component is selected from the group consisting of hypochlorous acid (sodium salt), iodine, lactic acid, acetic acid, peroxyacetic acid, and quaternary ammonium compounds.

5. The method according to claim 1, wherein said accessed quantity of EO ice further comprises an additional quantity of EO water containing said sanitizing compound therein.

6. The method according to claim 1 wherein said quantity of EO ice containing said sanitizing component therein comprises between 250 g and 1000 g of EO ice containing said sanitizing component therein.

7. A method of in-place sanitizing a contaminated meat grinder, comprising the steps of:
a. accessing a quantity of BO ice containing a sanitizing component frozen therein; and
b. exposing an operating meat grinder contaminated with E. coli O157: H7 to said quantity of EO ice for at least 3 minutes, thereby reducing said contamination of said meat grinder by E. coli O157: H7.

8. The method according to claim 7, wherein said sanitizing component is free chlorine at a concentration of between 50 to 200 mg/L.

9. The method according to claim 7, wherein BO ice has a hardness of between 75% and 100%, where said hardness is determined by measuring a latent heat capacity of said EO ice in Btu/lb divided by 144 Btu/lb and expressed as a percentage.

10. The method according to claim 1, wherein said sanitizing component is selected from the group consisting of hypochlorous acid (sodium salt), iodine, lactic acid, acetic acid, peroxyacetic acid, and quaternary ammonium compounds.

11. The method according to claim 1, wherein said accessed quantity of EO ice further comprises an additional quantity of EO water containing said sanitizing compound therein.

12. The method according to claim 1 wherein said quantity of EO ice continuing said sanitizing component therein comprises between 250 g and 1000 g of EO ice containing said sanitizing component frozen therein.
